(19) United States
(12) Patent Application Publication
(10) Pub. No.: US 2004/0142833 A1
(43) Pub. Date: Jul. 22, 2004

(54) Dual system soap and emulsifier cleansing method

(75) Inventor: Colin Frank Manzon, Harrison Township, MI (US)

Correspondence Address: COLIN F. MANZON, 25181 ST. CHRISTOPHER, HARRISON TOWNSHIP, MI, 48045, US

(73) Assignee: Colin Frank Manzon, Harrison Township, MI (US)

(21) Appl. No.: 10/248,409

(22) Filed: Jan. 16, 2003

(51) Int. Cl. A61K 7/50

(52) U.S. Cl. 510/141

(57) ABSTRACT

A twofold cleansing method for treating an oleaginous skin condition, which comprises of a dual action system, that when used simultaneously provides essential cleansing benefits for the epidermis. Although this process involves exact amounts of fats, oils, sodium hydroxide, essential oils, grains, vitamins, water, and plant extracts, other ingredients can be added to increase the effectiveness of this process. As well as fragrances may be added to make the scent more pleasant.

### CROSS REFERENCE TO RELATED APPLICATIONS

U.S. Pat. No. 5,096,709 March, 1992 Vandersloot U.S. Pat. No. 5,385,733 January, 1995 Mankovitz U.S. Pat. No. 5,529,784 June, 1996 DiPippo U.S. Pat. No. 5,894,020 April, 1999 Concha U.S. Pat. No. 6,218,348 April, 2001 Aronson, et al.

### BACKGROUND OF INVENTION

In general, all patents listed are for a customary bar of soap, in which the applicants either towel dry, or air-dry their skin and product usage is complete. In my patent, after applicant is dry, a secondary procedure is used, in which an emulsifier is applied topically to the skin to give it a healthy skin tone

### DETAILED DESCRIPTION

A person who has skin, which is oleaginous, first wets body. After in which the bar of soap, which contains a combination of melaleuca alternifolia (tea tree oil), oatmeal flour, vitamin E, and other soap products described in claim, is applied to the skin.

Then the soap product is put on a washcloth and circulated over entire body. The product is then left on for a minimum of 30 seconds to let active ingredients work. The combination of melaleuca alternifolia, which helps dry up oleaginous skin, oatmeal flour, which helps draw impurities from your skin, vitamin E, which is absorbed through the skin, and helps promote blood circulation, is accompanied by a lathering composition which includes, but not limited to coconut oil at 76 degree white, soybean shortening, sodium hydroxide, and animal fat.

Though there are similar patents on the market for the bar soap described above, E.G. U.S. Pat. No. 6,218,348, this product contains salts of protic acid, and polyalkylene glycol. My invention carries none of these elements. Another similar patent U.S. Pat. No. 5,894,020, contain Salicylic acid, and is mixed with soda, and a pot ash, this patent carries neither. Further more, this invention also adds oatmeal flour, which is not a benefaction agent in either patent.

Oatmeal itself has been well documented to help bring impurities out of your skin. In many studies, as provided by research doctors, that can even be found on WEBMD.com., oatmeal baths are prescribed for such ailments, but not limited to erythema infectiosum, chicken pox, and to help relieve the itching of chicken pox. Also health companies as accredited as Humana, have also posted articles on WEBMD.com, advising oatmeal as a source of good cleansing practice.

Although this product may be duplicated in many forms E.G. liquid, powder, etc. A solid bar of soap is most preferred. You can have the many benefits from my dual cleansing method, without having to add any creams and/or powders, after a normal washing.

After which, the second part of my system, in which no other patent on the market has, involves the applicant, who is then instructed to towel off body, and while pores are still open, they are to topically apply emulsifier provided. This agent is not to be wiped off but allowed to dry naturally on skin. (About 1-2 minutes) If desired, a second application is then performed with the emulsifier

The benefits shown in this procedure with this mixture of ingredients has been shown beneficial to skin appearance.

### SUMMARY OF INVENTION

The invention presented before you is a binary wash system in which the bar of soap does a deep thorough cleansing of the skin, and helps dry up oleaginous areas of the skin. However, by just doing this application alone the skin is left dry. A second procedure, in which the emulsifier is added after the skin is dry, produces a skin that is now moisturized and produces a healthy glow to it. The person using this treatment does not have to put any medications on separately; the results are achieved with normal washing procedures. The ingredients in this soap can be regulated by the amount of product used. Other ingredients may be added to the soap and/or emulsifier to increase its potency.

Added Benefits for Emulsifier

Although I plan to patent my product as listed, there are many beneficial additives that may be included in my invention. Listed below are some but not limited to:

Essential oils, fats, vegetable oils, waxes, plant derivatives, alcohols, vitamins, animal byproducts, fatty acids, salts, water, and sunscreen protection.

r

What is maintained is:

1. A solid bar of soap to deep clean and help eliminate an oleaginous skin presence.
An emulsifier to counteract the dryness of claim A, and moisturize the washing area.
Subject A comprising of:
Oatmeal flour in a quantity of at least 0.25% with a combination of oils, fats, essential oil, vitamins, water, and salts comprising the final ratio of elements to the bar.
The soap according to claim 1, whereas the composition is between 0.25% and 5%.
The soap according to claim 1, the said composition is at approximately 2%.
the soap according to claim 1, further includes coconut oil at 76 degree white.
The soap according to claim 4, whereas the composition is between 10% to 40%.
The soap according to claim 4 is at approximately 25%.
The soap according to claim 4, further includes Soybean shortening 100% pure.
The soap according to claim 7, whereas the composition is between 30% to 60%.
The soap according to claim 7, is at approximately 46%.
The soap according to claim 7, further includes melaleuca alternifolia (tea tree oil).
The soap according to claim 1 0, whereas the composition is between 0.5% to 10%.
The soap according to claim 1 0, is at approximately 3%.
The soap according to claim 10, further includes Vitamin E.
The soap according to claim 1 3, whereas the composition is between 0.25% to 5%.
The soap according to claim 1 3, is at approximately 1.5%.
The soap according to claim 13, further includes a combination of Sodium Hydroxide and water.
The soap according to claim 1 6, whereas the composition is between 5% to 30%.
The soap according to claim 1 6, is at approximately 13%.
The soap according to claim 1 6, further includes animal lard.
The soap according to claim 1 9, whereas the composition is between 5 to 30%.
The soap according to claim 1 9, is at approximately 16%.
The soap according to claim 1 9, whereas the treatment is followed up by an emulsifier.
The emulsifier according to claim 22, includes Aloe Vera in a gelatinized state, although, a liquid can also be used.
The emulsifier according to claim 22, whereas the composition is between 70% to 99%.
The emulsifier according to claim 22, is at approximately 93%.
The emulsifier according to claim 22, further includes Vitamin E, brought to a water-soluble state for absorption.
The emulsifier according to claim 26, whereas the composition is between 0.25% to 30%.
The emulsifier according to claim 26, is at approximately 7%.
The emulsifier according to claim 22, whereas a composition of less than 0.5% includes citric acid, vegetable gum, sodium benzoate, and potassium sorbate as a preservative.
